(19) United States
(12) Patent Application Publication
(10) Pub. No.: US 2026/0276545 A1
(43) Pub. Date: Sep. 17, 2026

(54) PORTABLE AUTOMATIC MICROSCOPY SYSTEMS FOR RAPID DETECTION OF MULTIPLE TYPES OF MICROPLASTICS

(71) Applicant: The Hong Kong University of Science and Technology, Hong Kong (CN)

(72) Inventors: Leung Yuk Frank LAM, Hong Kong (CN); Ka Sin LAM, Hong Kong (CN); Xuanming CHEN, Hong Kong (CN); Sze Ling CHEUK, Hong Kong (CN); Moyu HU, Hong Kong (CN)

(21) Appl. No.: 19/565,598

(22) Filed: Mar. 13, 2026

Related U.S. Application Data
(60) Provisional application No. 63/771,785, filed on Mar. 14, 2025.

(51) Int. Cl. G01N 21/64; G01N 33/18

(52) U.S. Cl. CPC G01N 21/643; G01N 21/6458; G01N 33/18; G01N 2201/023

(57) ABSTRACT

A portable automatic microscope system for on-site and real-time detection and classification of plastics and fibers in environmental and industrial water samples is provided. The system includes a sample introduction module for receiving water samples, a filtration module including a membrane holder and at least one membrane filter for retaining plastics and fibers, and a dye application module configured to apply a dye matrix containing at least one lipophilic fluorescent dye and at least one hydrophilic (water-soluble) colorimetric dye. A microscope imaging module includes an optical system with automatic focus adjustment, a controllable visible and ultraviolet illumination source, and a digital camera for capturing images of the retained plastics and fibers. A control module coordinates operation of the system components, while a computing module receives and analyzes the captured images to identify regions of interest, extract color, size, and morphology features, and classify plastics and fibers using image analysis methods.

### CROSS-REFERENCE TO RELATED APPLICATIONS

The present application claims priority from U.S. Provisional Utility Patent application No. 63/771,785 filed Mar. 14, 2025; the disclosure of which is incorporated herein by reference in its entirety.

### FIELD OF THE INVENTION

The present invention generally relates to the fields of characterization and detection technology. More specifically the present invention relates to the development of a portable, automated system for the rapid, on-site, and real-time detection and classification of plastics and fibers.

### BACKGROUND OF THE INVENTION

The growing global concern over plastic pollution in terrestrial and marine environments has driven the development of numerous techniques for detecting and analyzing microplastics and microfibers. Existing approaches for identifying and classifying plastics and fibers in environmental and industrial samples predominantly rely on manual inspection or laboratory-based analytical systems. These conventional methods are generally time-consuming, equipment-intensive, and unsuitable for rapid, large-scale, or on-site monitoring.

One widely adopted technique for microplastic detection is staining with Nile Red, a fluorescent dye that preferentially binds to hydrophobic materials, including many plastic polymers. Nile Red staining enables visualization of microplastics under fluorescence microscopy. For example, Maes et al., in “Nile red staining in microplastic analysis-proposal for a reliable and fast assessment method” (Environmental Monitoring and Assessment, 2017), reported an improved Nile Red staining protocol designed to enhance the speed and reliability of microplastic detection. Although this method offers advantages over traditional staining approaches, it remains dependent on laboratory workflows, manual handling, and post-processing analysis.

Another commonly used approach involves vibrational spectroscopy techniques, particularly Fourier-transform infrared (FTIR) spectroscopy and Raman spectroscopy, which identify microplastics based on characteristic molecular vibrational signatures. FTIR spectroscopy is typically applied to larger microplastic particles, while Raman spectroscopy, offering higher spatial resolution, is more suitable for smaller particles. Löder and Gerdts, in “Analysis of environmental microplastics by vibrational microspectroscopy: FTIR, Raman or both?” (Analytical and Bioanalytical Chemistry, 2015), compared these techniques and highlighted that a combined FTIR-Raman approach can improve identification accuracy. However, both methods suffer from significant practical limitations, including high equipment costs, complex operation, long analysis times, and reliance on skilled personnel.

Despite their analytical capabilities, existing microplastic detection technologies exhibit several major drawbacks. First, conventional methods are time-consuming and may require hours or days to complete, rendering them impractical for rapid or high-throughput screening. Second, most systems are laboratory-bound and lack portability, limiting their applicability for in-field monitoring in remote, marine, or industrial environments. Third, advanced spectroscopic instruments such as FTIR and Raman systems are expensive and operationally complex, restricting accessibility. Finally, many current techniques are retrospective in nature, requiring sample collection and off-site analysis, which delays the availability of actionable data and hinders real-time decision-making.

### SUMMARY OF THE INVENTION

It is an objective of the present invention to provide the systems and method to solve the aforementioned technical problems.

In accordance with a first aspect of the present invention, a portable automatic microscope system for detecting and classifying plastics and fibers in an environmental and/or industrial water sample is provided. Specifically, the portable automatic microscope system includes the following components: a sample introduction module configured to receive an environmental and/or industrial water sample; a filtration module including a membrane holder and at least one membrane filter configured to retain plastics and fibers from the water sample; a dye application module configured to deliver a dye matrix onto the retained plastics and fibers, in which the dye matrix includes at least one lipophilic fluorescent dye and at least one hydrophilic (water-soluble) colorimetric dye; a microscope imaging module, including a an optical system comprising an objective lens and a linear actuator or stepper motor for automatic focus adjustment, a controllable illumination source configured to provide visible light and ultraviolet light, and a digital camera configured to capture images of the retained plastics and fibers; a control module configured to control operation of the sample introduction module, filtration module, dye application module, and microscope imaging module; and a computing module including a processor and memory. The computing module being configured to receive the captured images from the digital camera; identify regions of interest corresponding to plastics and fibers; extract color, size, and morphology features from the regions of interest; and classify the plastics and fibers based on the extracted features using an image analysis method. It is worth noting that the system is configured to perform on-site and real-time detection and classification of plastics and fibers in the environmental and/or industrial water sample.

In accordance with one embodiment, the membrane holder is configured to receive the at least one membrane filter, and at least one membrane filter has a pore size within about 100 μm to about 5000 μm.

In accordance with another embodiment, the membrane filter is made of PVDF, cellulose acetate, or nylon.

In accordance with yet another embodiment, the portable automatic microscope system further includes a backwash module configured to clean the membrane filter.

In accordance with yet another embodiment, the system is operably connected to a computing device having a display, the computing device being configured to allow a user to operate and monitor the system.

In accordance with yet another embodiment, the sample introduction module includes a pump configured to deliver the water sample.

In accordance with yet another embodiment, the lipophilic fluorescent dye preferentially associates with hydrophobic polymer surfaces, optionally selected from Nile Red and/or structurally related lipophilic fluorophores.

In accordance with yet another embodiment, the hydrophilic (water-soluble) colorimetric dye includes one or more hydrophilic (water-soluble) dyes, optionally including Safranin T and Acid Blue 9.

In accordance with yet another embodiment, the computing module is further configured to generate and display a quantitative analysis report of detected plastics and fibers.

In accordance with yet another embodiment, the computing module comprises a convolutional neural network (CNN) configured to classify plastics and fibers.

In accordance with a second aspect of the present invention, a method for detecting and classifying plastics and fibers in an environmental and/or industrial water sample using a portable automatic microscope system is provided. The method includes the following steps: introducing an environmental and/or industrial water sample into the system; filtering the water sample through a membrane filter to retain plastics and fibers on the membrane filter; applying a dye matrix onto the retained plastics and fibers, in which the dye matrix includes at least one lipophilic fluorescent dye and at least one hydrophilic colorimetric dye; illuminating the retained plastics and fibers with visible light and/or ultraviolet light; capturing one or more images of the retained plastics and fibers using a digital camera; transmitting the captured images to a computing module; analyzing the captured images using the computing module to identify regions of interest corresponding to plastics and fibers, extract color, size, and morphology features from the regions of interest, and classify the plastics and fibers based on the extracted features and generate classification results; and outputting the classification results identifying types of plastics and/or fibers present in the environmental and/or industrial water sample.

In accordance with one embodiment, the filtering step includes passing the water sample through a membrane filter selected from a plurality of replaceable membrane filters having a pore size within about 100 μm to about 5000 μm. In accordance with yet another embodiment, the dye matrix is dissolved

in a solvent system including water and an organic solvent having one or more of methanol, ethanol, acetone, and dimethyl sulfoxide.

In accordance with yet another embodiment, the method further includes adjusting the pH value of the dye matrix to approximately 9-14 prior to applying the dye matrix to the retained plastics.

In accordance with yet another embodiment, the method further includes heating the dye matrix and retained plastics to a temperature of 25° C. to 80° C. for 5 to 300 minutes before the illuminating.

In accordance with yet another embodiment, the classifying step includes distinguishing cotton fibers, polyester fibers, polyamide 6, polyamide 66, polypropylene, acrylonitrile butadiene styrene, polymethyl methacrylate, and polyvinyl chloride.

In accordance with yet another embodiment, the method further includes cleaning the membrane filter by backwashing after image capture.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Embodiments of the invention are described in more details hereinafter with reference to the drawings, in which:

FIG. 1 depicts the design of a portable automatic microscope system prototype in according to one embodiment of the present invention;

FIG. 2 depicts the preliminary result of cotton fiber (black) and polyester fiber (blue);

FIGS. 3A-3B the staining result in pH 10 heating test of 6 types of plastic under visible light (FIG. 3A) and under 254 nm UV light (FIG. 3B); and

FIG. 4 depicts the structural block diagram of a portable automatic microscope system prototype in according to one embodiment of the present invention.

### DETAILED DESCRIPTION

In the following description, systems and/or methods of detecting and classifying plastics and fibers in an environmental and/or industrial water sample and the likes are set forth as preferred examples. It will be apparent to those skilled in the art that modifications, including additions and/or substitutions may be made without departing from the scope and spirit of the invention. Specific details may be omitted so as not to obscure the invention; however, the disclosure is written to enable one skilled in the art to practice the teachings herein without undue experimentation.

The present invention provides an innovative and practical solution for the identification and classification of plastics and fibers from a wide range of sources, including industrial discharge, environmental water samples, and marine microplastic pollution. By combining an advanced dye-staining method with a portable automatic microscope system, the invention enables rapid, accurate, and cost-effective detection of microplastics and microfibers directly at the point of sampling. The system is specifically designed to support on-site and real-time analysis, thereby eliminating the need for complex laboratory infrastructure and significantly reducing testing time and operational costs.

In particular, the invention introduces a novel staining method that employs a special dye matrix configured to interact with the unique surface properties and chemical characteristics of different plastics and fibers. Through controlled testing conditions, such as pH adjustment, heating, and dual-mode illumination, the dye matrix produces distinct colorimetric and fluorescent responses for different polymer types and fiber materials. This enables precise differentiation and classification of plastics and fibers, even in complex environmental and industrial matrices. When integrated with the portable automatic microscope, the staining method allows for immediate visualization, image capture, and automated classification of contaminants in diverse sample types.

The portable and automated design of the system facilitates large-area detection and rapid screening in marine, coastal, industrial, and wastewater environments. This makes the invention particularly well-suited for applications in marine microplastic pollution monitoring, industrial emission surveillance, environmental impact assessments, and sustainability programs. By enabling field-based analysis, the system provides regulatory authorities, environmental agencies, and industrial operators with a powerful tool for real-time compliance monitoring and pollution control.

Moreover, the invention offers a practical technological foundation for establishing and enforcing new plastic emission standards in regions where regulatory frameworks are currently limited or under development. The ability to rapidly detect and classify plastic pollutants on-site supports evidence-based policymaking, improves enforcement efficiency, and promotes environmentally responsible industrial practices. Overall, the invention delivers a faster, more efficient, and highly deployable detection platform that contributes meaningfully to global efforts in environmental protection, pollution mitigation, and sustainable development.

As used herein, the term “lipophilic fluorescent dye” refers to a fluorescent compound having a higher affinity for hydrophobic or non-polar materials than for aqueous phases, such that the dye preferentially associates with, adsorbs onto, or partitions into hydrophobic polymeric materials, including plastic particles and synthetic polymer surfaces (e.g., synthetic fibers), when present in an aqueous or mixed solvent system. Upon excitation by light of an appropriate wavelength, the lipophilic fluorescent dye emits fluorescence that is detectable by an optical imaging system. In some embodiments, the lipophilic fluorescent dye also provides a detectable optical contrast under visible illumination when associated with hydrophobic polymeric materials.

In the context of the present invention, the lipophilic fluorescent dye is capable of providing fluorescence contrast for polymeric particles and/or synthetic fibers by preferential association with relatively hydrophobic domains on polymer chains and/or polymer surfaces, due to hydrophobic interactions, π-π interactions, or van der Waals forces between the dye molecules and polymer chains and polymer surfaces of plastics. Non-limiting examples include Nile Red, BODIPY derivatives, and other hydrophobic fluorophores capable of emitting fluorescence under visible or ultraviolet excitation.

As used herein, the term “hydrophilic colorimetric dye” refers to a water-soluble dye that preferentially remains in the aqueous phase under the staining conditions and comprises one or more polar and/or ionizable groups that enable interaction with hydrophilic fibers, hydrophilic surfaces, and/or polar functional groups present on retained matter (e.g., polar regions on polymer or fiber surfaces). In addition to such polar and/or ionizable group(s), the hydrophilic colorimetric dye may comprise an aromatic and/or other relatively hydrophobic chromophore moiety (e.g., phenyl-containing structures) that can associate with polymeric materials via hydrophobic and/or π-π interactions; however, the polar and/or ionizable group(s) confer increased selectivity toward retained matter presenting polar and/or ionizable functional groups and/or relatively more polar polymer surfaces as compared with non-polar plastics (e.g., the dye may preferentially associate with microplastics having a higher density of polar bonds and/or polar surface functionalities). Non-limiting examples include Safranin T, Acid Blue 9, and other water-soluble dyes capable of producing distinct color contrasts under visible illumination.

As used herein, “visible illumination” refers to broadband visible light (e.g., white light) applied for reflectance- and/or transmittance-based imaging to obtain absorbance- and/or color-based contrast. Although broadband visible illumination may include wavelengths capable of exciting fluorophores, the imaging configuration is selected such that the captured signal is predominantly reflectance and/or transmittance rather than fluorescence. In contrast, “visible excitation” refers to illumination at a selected wavelength or a band-limited portion of the visible spectrum (e.g., selectable within about 400-800 nm, optionally a narrow band centered at a chosen wavelength) applied for fluorescence imaging, typically in combination with excitation and emission filters such that the detected signal is predominantly fluorescence emission.

In some embodiments, the hydrophilic colorimetric dye provides color-contrast features in images captured under visible illumination (e.g., broadband white light for reflectance- and/or transmittance-based imaging, with the captured signal predominantly non-fluorescent) associated with polar and/or ionizable functional groups present on retained matter. In some embodiments, under ultraviolet excitation and/or visible excitation used for fluorescence imaging, the hydrophilic colorimetric dye provides a distinct optical response and/or modifies the apparent fluorescence response of the retained matter (e.g., by spectral overlap, energy transfer, and/or quenching).

In the present invention, the hydrophilic colorimetric dye may be applied as a standalone stain and/or as a pre-stain prior to application of the lipophilic fluorescent dye, to impart a color-contrast pattern biased toward polar and/or ionizable sites on retained matter under visible illumination, and, in some embodiments, to modulate the composite optical response under ultraviolet and/or visible excitation when subsequently used together with the lipophilic fluorescent dye.

In some embodiments, the lipophilic fluorescent dye and the hydrophilic colorimetric dye are used together as a dye matrix. The lipophilic fluorescent dye preferentially provides fluorescence contrast by staining non-polar polymer domains, while the hydrophilic colorimetric dye preferentially provides color contrast by staining polar functional groups, and in some embodiments interacts with the lipophilic fluorescent dye to further differentiate optical responses under visible illumination and ultraviolet and/or visible excitation. This combined dye-matrix approach enables simultaneous detection and classification using a single imaging workflow.

In some embodiments, differentiation and/or classification of retained matter is achieved by interpreting a combination of image features obtainable under multiple imaging conditions, regardless of whether a lipophilic fluorescent dye, a hydrophilic colorimetric dye, or a mixture thereof is used. Such image features can include, without limitation: (i) color and/or intensity contrast under visible illumination, (ii) fluorescence response under ultraviolet excitation, (iii) fluorescence response under visible excitation, and (iv) morphology- and size-related features (e.g., particle shape, aspect ratio, texture, and size distribution). In some embodiments, classification is based on a multi-feature interpretation rather than a single cue alone.

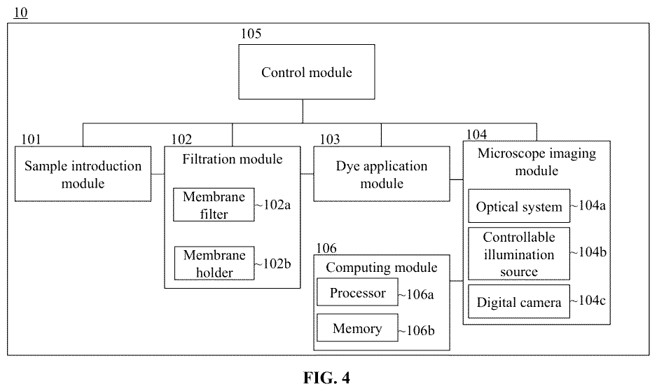

In accordance with a first aspect of the present invention, a portable automatic microscope system for detecting and classifying plastics and fibers in an environmental and/or industrial water sample is provided. The system is configured to enable on-site and real-time analysis, thereby allowing rapid identification and classification of microplastics and microfibers directly at the sampling location without the need for laboratory-based equipment. As shown in FIG. 4, the system 10 includes a sample introduction module 101 configured to receive an environmental and/or industrial water sample, a filtration module 102, a dye application module 103, a microscope imaging module 104, a control module 105, and a computing module 106.

The sample introduction module 101 is configured to receive the environmental and/or industrial water sample and may comprise a pump (not shown in figure) for delivering the water sample into the system 10. In some embodiments, the pump is configured to provide controlled and continuous flow of the water sample from a sample container, pipeline, or collection vessel into the filtration module 102. The filtration module 102 includes a membrane holder 102b configured to receive a selected one of a plurality of replaceable membrane filters 102a for retaining plastics and fibers from the water sample while allowing the liquid phase to pass through. In some embodiments, the membrane filter 102a has a pore size ranging from 100 μm to 5000 μm, enabling effective capture of plastics and fibers of various sizes. The membrane filter 102a may be made of PVDF, cellulose acetate, or nylon, thereby providing chemical compatibility, mechanical strength, and reliable filtration performance. The filtration module 102 may further be operatively coupled to a backwash module (not shown in figure) configured to clean the membrane filter 102a after analysis by reversing the flow of clean fluid through the membrane, thereby removing retained debris and preparing the membrane for subsequent use.

The dye application module 103 is configured to deliver a dye matrix onto the plastics and fibers retained on the membrane filter 102a. The dye matrix includes at least one lipophilic fluorescent dye and at least one hydrophilic (water-soluble) colorimetric dye, enabling both fluorescence-based and color-based visualization of plastics and fibers. In some embodiments, the lipophilic fluorescent dye includes Nile Red, which selectively stains hydrophobic polymeric materials. The colorimetric dye may include one or more of Safranin T and Acid Blue 9, which provide distinct color signatures when interacting with different types of fibers or plastics. The dye application module 103 may be configured to dispense a predetermined volume of the dye matrix onto the membrane filter 102a in an automated and controlled manner to ensure uniform staining of the retained plastics and fibers.

The microscope imaging module 104 is configured to image the stained plastics and fibers retained on the membrane filter 102a. The microscope imaging module 104 includes an optical system 104a including an objective lens and a linear actuator or stepper motor for automatic focus adjustment, thereby enabling precise and repeatable focusing without manual intervention. The microscope imaging module 104 further includes a controllable illumination source 104b configured to provide visible light and ultraviolet light, allowing imaging under different illumination conditions to enhance contrast and reveal dye-specific fluorescence or colorimetric responses. A digital camera 104c is operatively coupled to the optical system 104a and configured to capture images of the retained plastics and fibers. In some embodiments, the digital camera 104c is a high-resolution camera capable of capturing still images and/or video streams of the stained membrane surface.

The control module 105 is configured to control operation of the sample introduction module 101, the filtration module 102, the dye application module 103, and the microscope imaging module 104. The control module 105 may include a microcontroller, programmable logic controller, or other control circuitry programmed to execute predefined sequences for sample delivery, filtration, dye application, illumination control, focus adjustment, and image capture. Through coordinated control of these modules, the system 10 is capable of performing automated, repeatable detection procedures with minimal user intervention.

The system further includes a computing module 106 including a processor 106a and memory 106b. The computing module 106 is configured to receive the captured images from the digital camera 104c and to perform image analysis on the received images. In operation, the computing module 106 identifies regions of interest corresponding to plastics and fibers in the images, extracts color, size, and morphology features from the regions of interest, and classifies the plastics and fibers based on the extracted features using an image analysis method. In some embodiments, the computing module 106 includes a CNN configured to classify plastics and fibers based on learned visual patterns, color signatures, and morphological characteristics. The computing module 106 may further be configured to generate and display a quantitative analysis report of detected plastics and fibers, including counts, size distributions, material classifications, and spatial distributions.

In some embodiments, the portable automatic microscope system 10 is further connected to a computing device with a display, such as a laptop computer, tablet, or embedded display, to allow a user to operate and monitor the system 10. Through the display, the user may view live images, adjust operational parameters, initiate analysis sequences, and review classification results and quantitative reports. The computing module 106 may be integrated within the system housing or may be implemented as an external computing device communicatively coupled to the system via wired or wireless connections.

Through integration of the sample introduction module 101, filtration module 102, dye application module 103, microscope imaging module 104, control module 105, and computing module 106, the system 10 is configured to perform on-site and real-time detection and classification of plastics and fibers in environmental and/or industrial water samples. The combined use of fluorescent and colorimetric dyes, automated imaging, and advanced image analysis enables rapid, accurate, and reproducible identification of plastics and fibers, making the system particularly suitable for applications such as environmental monitoring, industrial wastewater analysis, marine pollution assessment, and microplastic surveillance.

In accordance with a second aspect of the present invention, a method for detecting and classifying plastics and fibers in an environmental and/or industrial water sample using a portable automatic microscope system is provided. In operation, an environmental and/or industrial water sample is first introduced into the system through a sample introduction interface. The introduced water sample is then directed to a filtration module, where the sample is passed through a membrane filter so that plastics and fibers present in the water are retained on the surface of the membrane filter while the liquid phase is discharged. In some embodiments, the filtering step comprises passing the water sample through a membrane filter selected from a plurality of replaceable membrane filters having pore sizes within about 100 μm to about 5000 μm, thereby balancing retention of microplastics/microfibres with throughput suitable for automated field operation.

After filtration, a dye matrix is applied directly onto the retained plastics and fibers on the membrane filter. The dye matrix comprises at least one lipophilic fluorescent dye and at least one hydrophilic (water-soluble) colorimetric dye. In preferred embodiments, the dye matrix comprises Nile Red as the lipophilic fluorescent dye and Safranin T and Acid Blue 9 as the water-soluble colorimetric dyes. The dye matrix is dissolved in a solvent system comprising water and one or more organic solvents selected from methanol, ethanol, acetone, DMSO, or any combinations thereof, thereby ensuring adequate solubility, penetration, and interaction between the dyes and the retained plastics and fibers. In some embodiments, prior to applying the dye matrix to the retained plastics, the pH value of the dye matrix is adjusted to approximately 9-14, such as by addition of an alkaline reagent, in order to enhance selective interaction between the dyes and plastic materials. In further embodiments, the dye matrix and the retained plastics are heated to a temperature ranging from 25° C. to 80° C. for a duration of 5 to 300 minutes before illumination, thereby promoting dye diffusion, adsorption, and binding to the plastic surfaces to improve staining contrast and classification accuracy.

Following dye application and optional pH adjustment and heating, the retained plastics and fibers are illuminated using visible light and/or ultraviolet light generated by a controllable illumination source of the portable automatic microscope system. One or more images of the retained plastics and fibers are then captured using a digital camera integrated with the microscope imaging module. The captured images are transmitted to a computing module comprising a processor and memory. The computing module analyzes the captured images to identify regions of interest corresponding to plastics and fibers, extract color, size, and morphology features from the regions of interest, and classify the plastics and fibers based on the extracted features using an image analysis method, such as a machine learning or convolutional neural network-based algorithm. The computing module then generates classification results identifying types of plastics and/or fibers present in the environmental and/or industrial water sample and outputs the classification results to a user interface, display, or external device.

In certain embodiments, the classifying step comprises distinguishing among cotton fibers, polyester fibers, polyamide 6 (PA6), polyamide 66 (PA66), polypropylene (PP), acrylonitrile butadiene styrene (ABS), polymethyl methacrylate (PMMA), and polyvinyl chloride (PVC) based on characteristic color signatures, fluorescence responses, and morphological features observed under visible and/or ultraviolet illumination. After image capture and analysis are completed, the method further comprises cleaning the membrane filter by backwashing, wherein a reverse flow of clean fluid is applied to the membrane filter to remove retained particles and residual dye, thereby preparing the system for subsequent sample analysis and ensuring consistent performance across multiple testing cycles.

Through the combination of controlled filtration, multi-dye staining, pH and thermal conditioning, dual-mode illumination, automated imaging, and intelligent image analysis, the method enables on-site and real-time detection and classification of plastics and fibers in environmental and/or industrial water samples with high sensitivity, specificity, and operational efficiency.

### EXAMPLES

### Example 1. Development of Portable Automatic Microscope System

The present invention presents a novel portable automatic microscope system designed for the on-site detection and classification of plastics and fibers. The system enables real-time identification of microplastics and fibers from environmental and industrial samples, especially laundry wastewater and marine water samples. The system integrates a staining module, a portable microscope with an image capture system, an autosampler for automatic liquid handling, and an AI-based image processing unit for material classification. Below, the components and operational steps of the system are described in detail.

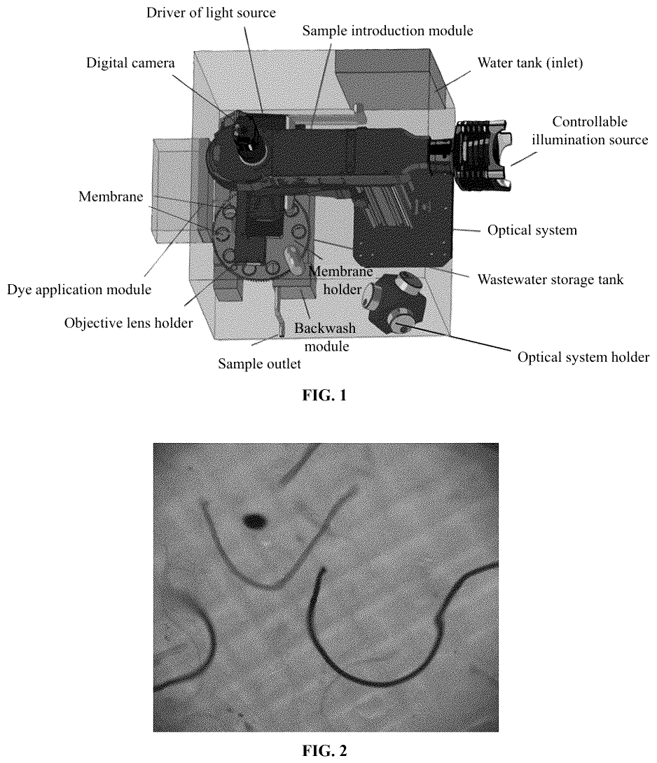

The system enables on-site and real-time analysis of microplastics and microfibers from environmental and industrial samples. The system integrates several core components as shown in FIG. 1, including the microscope imaging module, autosampler, controller, and computing module. Each component is described in detail below to ensure the completeness and sufficiency of disclosure.

The microscope imaging module serves as the central unit for imaging, optical detection, and analysis of plastics and fibers. It incorporates an optical system including a microscope objective and an adjustable focus holder. The optical system includes a high-magnification variable-focus objective lens capable of magnifications ranging from 10× to 1500×, enabling high-resolution imaging of microplastics and fibers. Focus adjustment is performed using a linear actuator or stepper motor controlled by the computing module, thereby enabling automatic focusing and improved image clarity. The field of view of the objective lens ranges from 30° to 120°, providing a wide observation area for capturing microplastics and fibers retained on a membrane filter.

The microscope imaging module further includes a lighting system featuring an adjustable LED light source with a wavelength range from 200 nm to 800 nm, supporting both visible and near-ultraviolet illumination. The light source provides a maximum brightness of approximately 1000 lumens to ensure sufficient illumination during imaging. Light intensity is controlled using a pulse width modulation (PWM) dimming system, allowing precise and dynamic control. The lighting system is capable of operating in multiple illumination modes, including continuous, pulsed, and strobed modes, to optimize image contrast and clarity for different sample types and detection requirements.

A light controller is provided to enable real-time adjustment of light intensity and switching between illumination modes. The light controller is integrated with the computing module and connected to the software interface, allowing users to adjust lighting parameters through a graphical user interface (GUI). In certain embodiments, the light controller is configured to automatically adjust illumination settings based on sample characteristics or image analysis feedback.

The microscope imaging module further includes a high-resolution digital camera having a resolution of at least 1920×1080 pixels and a frame rate of 30 to 60 frames per second. The camera is connected to the processing unit via a high-speed interface such as USB 3.0, USB-C, or wireless communication. The camera supports automatic and manual exposure control to optimize imaging under different lighting conditions. During operation, the camera captures high-resolution images of the membrane filter, which are subsequently processed using AI-based image analysis algorithms.

The autosampler is configured to automatically introduce samples, perform staining, and manage filtration, and includes a sample introduction module, a filtration module and a dye application module. The sample introduction module allows wastewater or other liquid samples to be introduced into a sample chamber. The sample introduction module is connected to a pump, such as a peristaltic pump, diaphragm pump, or solenoid pump, to regulate sample flow. The flow rate is controllable in the range of approximately 10 mL/min to 600 mL/min to accommodate different sample volumes and analysis requirements. The sample introduction module also includes a water outlet discharges the processed sample after filtration, staining, and analysis.

The dye application module including a dye injector with precision nozzles capable of delivering dye volumes in the range of approximately 1 mL to 50 mL. Dye injection is automated and controlled using a stepper motor-driven syringe pump, providing an injection accuracy of approximately +0.1 mL. The system supports injection of multiple dyes either simultaneously or sequentially according to a predefined detection protocol, thereby enabling selective staining of plastics and fibers.

The filtration module includes a membrane holder and one or more membrane filters, and the membrane holder houses a selected one of a plurality of replaceable membrane filters for capturing microplastics and microfibers from the sample. The membrane filter may be formed of PVDF, cellulose acetate, nylon, or other suitable materials, and may have a pore size within about 100 μm to about 5000 μm. The membrane holder may be configured to promote uniform fluid distribution across the membrane surface and to reduce clogging during operation.

The autosampler further includes a backwash system configured to clean the membrane after each analysis cycle. Backwashing is performed by reversing the flow of clean water to remove retained particles and debris from the membrane surface. This cleaning process extends membrane service life and prepares the system for subsequent analyses.

The system includes a control module including a microcontroller, an embedded controller, and/or a programmable logic controller (PLC). In some embodiments, the control module includes an Arduino, a Raspberry Pi, or an industrial PLC. The control module is configured to execute pre-programmed control sequences for operating pumps, valves, actuators, the illumination system, and the camera. The control module communicates with system components using one or more serial communication protocols including, but not limited to, I²C, SPI, UART, CAN, or Ethernet.

The system further includes a computing module implemented on a connected computer, industrial PC, or embedded processor. The computing module is configured to provide a graphical user interface on a display for controlling focus, zoom, light intensity, and image exposure, and for monitoring sample loading, dye application, filtration, and image acquisition processes. The computing module is further configured to execute AI-based image processing algorithms to automatically identify and classify plastics and fibers based on color, morphology, size, and shape features. Captured images are processed to identify regions of interest, classify polymer types including but not limited to PET, PP, and ABS, and generate an analysis report for user review.

In some embodiments, the control module and the computing module are operatively coupled and configured to exchange control signals and data to coordinate fluid handling, imaging, and analysis operations.

In operation, a wastewater sample, typically having a volume in the range of about 50 mL to 500 mL, is introduced into the system through a water inlet. The sample is driven through the inlet by a pump, such as a peristaltic pump or a diaphragm pump, operating at a controlled flow rate of approximately 10 to 600 mL per minute. The sample is directed into a membrane holder containing a membrane filter selected from a plurality of replaceable membrane filters, each having a pore size within about 100 μm to about 5000 μm. As the sample passes through the membrane, microplastics and fibers larger than the pore size are retained on the surface of the membrane, while the filtered water is discharged through a water outlet.

Following filtration, a dye matrix is injected into the membrane holder using an automated precision syringe pump with a volume accuracy of approximately +0.1 mL. The total volume of the dye matrix introduced may range from about 1 mL to 100 mL. The dye matrix comprises 50% to 99% by volume of component A, and 0.5% to 50% by volume of components B and C, respectively. Component A is preferably Nile Red, but may also include other functionally equivalent dyes, including organic dyes or fluorescent dyes. Components B and C are preferably Safranin T and Acid Blue 9, respectively, but may alternatively be selected from xanthene dyes, phthalocyanine dyes, quinoline dyes, or other functionally equivalent dyes. The dye matrix has a concentration ranging from about 0.1 mmol/L to 10 mol/L.

The dye matrix is prepared in a solvent system comprising a mixture of water and one or more organic solvents, wherein the organic solvent is selected from methanol, ethanol, acetone, DMSO, or combinations thereof, and wherein the volumetric ratio of organic solvent to water ranges from approximately 1:99 to 99:1. For fiber detection, a well-mixed dye solution having a volume of about 1 mL to 20 mL is directly dispensed onto the fiber-containing region and incubated for approximately 10 to 30 seconds. For plastic detection, the pH of the dye solution is adjusted to a value in the range of about 9 to 14 using an alkali selected from potassium hydroxide (KOH), sodium hydroxide (NaOH), calcium hydroxide (Ca(OH)₂), or other functionally equivalent bases. The mixture of plastics and dye solution is then heated to a temperature of approximately 25° C. to 80° C. for a duration of about 5 to 300 minutes to enhance interaction between the dye and the microplastics prior to detection under ultraviolet and visible light in the wavelength range of approximately 200 nm to 800 nm.

After completion of the dyeing process, the membrane filter is positioned within the field of view of the microscope, and a digital camera integrated with the microscope captures one or more images of the membrane surface. In one embodiment, the camera has a resolution of approximately 1920×1080 pixels and operates at a frame rate in the range of about 30 to 60 frames per second. The captured images are automatically transmitted to a connected artificial intelligence (AI)-based image analysis system via a data interface such as USB 3.0, USB-C, or a wireless connection including Wi-Fi. The images may be stored in standard digital image formats, including but not limited to TIFF, JPEG, or PNG, for subsequent processing and analysis.

The AI-based image analysis system processes the captured images using machine learning algorithms, including convolutional neural networks (CNNs). The system automatically identifies one or more regions of interest (ROI) within each image and performs image segmentation to divide the ROI into color-coded regions based on the detected dye signals. Each pixel or group of pixels is assigned a color code corresponding to a specific material type, for example, blue for polyvinyl chloride (PVC) or orange for nylon-6 (NA6) under 254 nm ultraviolet illumination.

Based on the segmented image data and color-coded information, the system classifies the detected particles as polyester, acetate, polymethyl methacrylate (PMMA), acrylonitrile butadiene styrene (ABS), PVC, or other material types using a pre-trained classification model.

### Example 2. Detection and Classification of Cotton and Polyester Microfibers Using the Portable Automatic Microscope System

The performance of the portable automatic microscope system is evaluated for the detection and classification of cotton and polyester microfibers in an aqueous sample using a specially formulated dye matrix including Nile Red, Safranin T, and Acid Blue 9. This example demonstrates the capability of the system to provide rapid, reliable, and real-time analysis of mixed fiber samples, in contrast to conventional manual microscopy-based detection methods.

A mixed fiber sample is prepared by dispersing 10 mg of polyester microfibers and 10 mg of cotton microfibers in 100 mL of water to form a homogeneous suspension. The dye matrix used for staining is prepared at a concentration of 1 mmol/L in a total volume of 10 mL. The solvent system includes a mixture of water and methanol in a volume ratio of 9:1. The dye matrix includes Nile Red as Component A at 95% by molar quantity, Safranin T as Component B at 2.5% by molar quantity, and Acid Blue 9 as Component C at 2.5% by molar quantity.

The experiment is conducted using the portable automatic microscope system, which includes an optical imaging system, an adjustable LED light source, a light controller, and a high-resolution digital camera. The system further includes an autosampler for automated sample and dye injection, a membrane holder equipped with at least one membrane filter having a pore size of 100 μm, a backwash system for membrane cleaning, and a control and image analysis software platform for automated operation and data processing.

The prepared 100 mL fiber suspension is introduced into the system through the water inlet. The sample is driven through the system by a pump and filtered through the membrane filter, thereby retaining the cotton and polyester microfibers on the surface of the membrane while allowing the filtrate to exit through the water outlet. After filtration, 5 mL of the dye matrix is withdrawn from the 10 mL stock solution using the autosampler and injected into the membrane holder. The dye solution is allowed to uniformly distribute over the retained microfibers. The stained fibers are incubated for approximately 15 seconds to allow sufficient interaction between the dye components and the fibers.

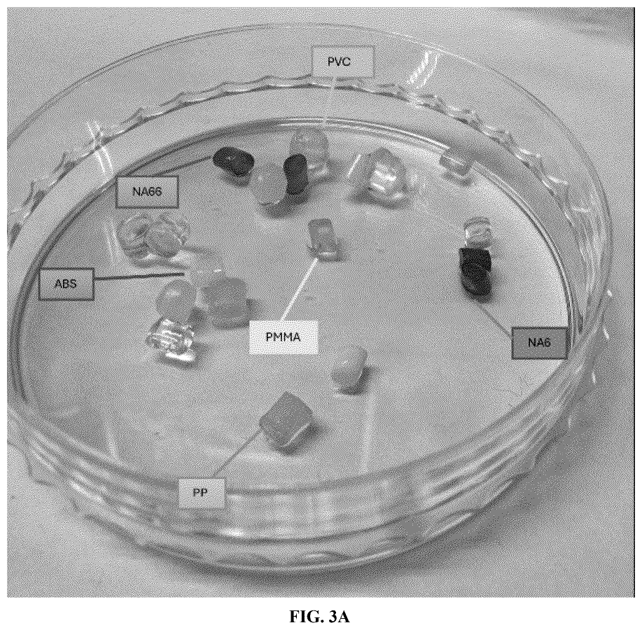

Following the staining process, the membrane filter is positioned under the microscope, and an image of the stained fibers is captured using the high-resolution camera at a resolution of 1920×1080 pixels, as shown in FIG. 2. The captured image is automatically transmitted to the control software for further processing and analysis. The AI-based image analysis module identifies regions of interest within the image and classified the fibers based on color and morphological features. In this example, cotton fibers appear as black regions, while polyester fibers appear as blue regions, enabling clear differentiation between the two fiber types. After completion of image acquisition and analysis, the system initiates a backwash process using clean water supplied in a reverse flow direction to remove residual fibers and dye from the membrane surface. The backwash process is carried out for approximately 30 seconds to 1 minute, after which the membrane is clean and the system is ready for subsequent sample analysis.

The analysis results demonstrate that the portable automatic microscope system achieves highly accurate classification of both cotton and polyester microfibers. In the analyzed samples, cotton fibers are identified with an accuracy of 100%, and polyester fibers are likewise identified with an accuracy of 100%, indicating that the combined use of the multi-component dye matrix and the AI-based image analysis module enables precise differentiation between natural and synthetic fibers.

The entire analytical workflow, including sample filtration, dye application, incubation, image capture, AI-based image analysis, and membrane cleaning by backwashing, is completed within approximately five minutes. This short processing time highlights the capability of the system to perform rapid, real-time analysis, which is a significant improvement over conventional manual microscopy and laboratory-based methods that typically require substantially longer processing times.

FIG. 2 illustrates a representative image captured by the system following staining and imaging. In the image, cotton fibers appear as black regions, while polyester fibers appear as blue regions, providing clear visual contrast and enabling reliable automated classification by the image processing algorithm.

This demonstrates that the portable automatic microscope system is capable of rapidly and accurately detecting and classifying microfibers in aqueous samples. The integration of the AI-based image analysis module with the specially formulated dye matrix enables automated, high-throughput detection with minimal user intervention. The ability to complete the entire detection and classification process within minutes significantly enhances operational efficiency and makes the system particularly suitable for on-site environmental monitoring, industrial wastewater analysis, and real-time pollution control applications.

### Example 3. Detection and Classification of Six Types of Plastics Using The Portable Automatic Microscope System

The effectiveness of the portable automatic microscope system in detecting and classifying six different types of plastics is evaluated using a specially formulated dye matrix composed of Nile Red, Safranin T, and Acid Blue 9. The experiment illustrates that, after exposure to alkaline conditions, heating, and ultraviolet (UV) illumination, each plastic type exhibits a distinct color response, enabling accurate visual and automated classification. The evaluated plastics include PA6, PA66, PMMA, PP, ABS, and PVC.

Each plastic sample is provided in an amount of 50 mg. The dye matrix is prepared to have a concentration of 5 mmol/L in a total volume of 100 mL, using a solvent system comprising water and methanol in a volume ratio of 19:1. The dye matrix consisted of Nile Red as Component A in an amount of 90-95% of the total molar quantity, Safranin T as Component B in an amount of 2.5-5% of the total molar quantity, and Acid Blue 9 as Component C in an amount of 2.5-5% of the total molar quantity.

The experiment is performed using the portable automatic microscope system equipped with an optical imaging system, an LED light source, a light controller, and a high-resolution digital camera. The system further includes an autosampler for automated liquid handling, a membrane holder containing a 100 μm pore-size membrane filter, a backwash system for membrane cleaning, and a computing module executing control software for automated operation and AI-based image analysis.

For staining, the six plastic samples (PA6, PA66, PMMA, PP, ABS, and PVC, 50 mg each) are placed into a reaction container. The prepared dye matrix solution (100 mL) is added to the container such that all plastic particles are fully submerged. The pH of the dye solution is adjusted to approximately 10 using potassium hydroxide (KOH). The reaction container is then placed in a heating unit and maintained at a temperature of 50° C. for a reaction time of 15 minutes to enhance dye-polymer interactions. Upon completion of the reaction, the entire mixture, including the dye solution and the stained plastic particles, is transferred directly to the filtration system without washing.

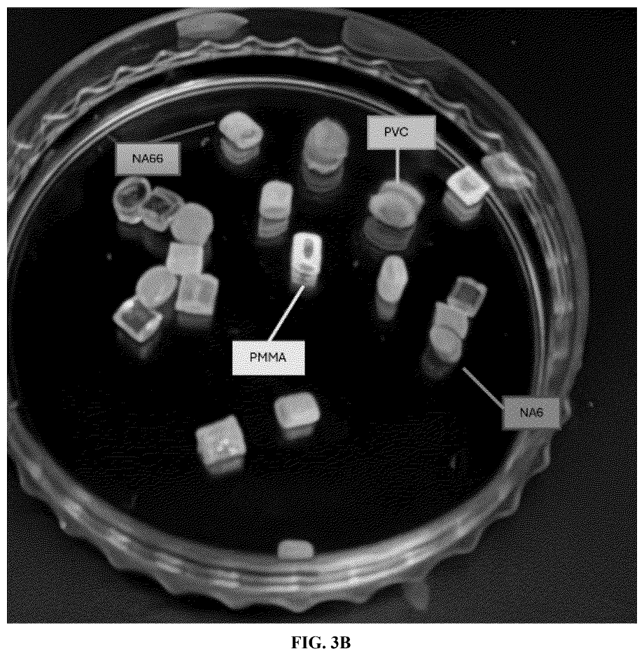

The mixture is introduced into the membrane holder and filtered through the 100 μm membrane filter. The membrane retains all stained plastic particles, while the dye solution is discharged through the system outlet. After filtration, the membrane containing the stained plastics is positioned under the portable automatic microscope. High-resolution images are captured under both visible light and UV light (254 nm), as illustrated in FIGS. 3A and 3B. The camera operates at a resolution of 1920×1080 pixels and a frame rate of 30-60 frames per second. The captured images are transmitted to a connected computing module for further processing.

The images are analyzed using an AI-based image analysis system. The system automatically identifies regions of interest corresponding to individual plastic particles and extracts color, size, and shape features from each region. Based on the unique color signatures produced by the dye matrix under visible and UV illumination, the AI system classifies each particle as PA6, PA66, PP, ABS, PMMA, or PVC.

Following image acquisition and analysis, the system initiats a backwash process to clean the membrane filter. Clean water is flushed through the system in reverse flow, removing residual particles from the membrane within approximately 30 seconds to 1 minute, thereby preparing the system for subsequent analysis.

The results show clear and distinct color responses for each plastic type. PA6 exhibits a strong red color under visible light, PA66 appears red under visible light, PP appears dim pink under visible light, ABS appears slight pink under visible light, PMMA exhibits a yellow color under UV light, and PVC exhibits a blue color under UV light. FIG. 3A shows representative images of PA6, PA66, PMMA, PP, and ABS under visible light, while FIG. 3B shows a representative image of PVC under UV illumination at 254 nm.

The total processing time, including dye application, pH adjustment, heating, filtration, image capture, image analysis, and membrane cleaning, is completed in less than 30 minutes.

This example demonstrates that the portable automatic microscope system, in combination with the multi-component dye matrix, can accurately and efficiently detect and classify multiple types of plastics based on colorimetric responses. The integration of chemical staining, automated filtration, high-resolution imaging, and AI-based classification enables rapid, reliable, and cost-effective plastic identification. The method is particularly suitable for on-site environmental monitoring, industrial wastewater analysis, and large-scale screening of plastic contamination.

The functional units and modules of the portable automatic microscope systems and/or the application methods in accordance with the embodiments disclosed herein may be implemented using computing devices, computer processors, or electronic circuitries including but not limited to application specific integrated circuits (ASIC), field programmable gate arrays (FPGA), microcontrollers, graphical processing units (GPU), and other programmable logic devices configured or programmed according to the teachings of the present disclosure. Computer instructions or software codes running in the computing devices, computer processors, or programmable logic devices can readily be prepared by practitioners skilled in the software or electronic art based on the teachings of the present disclosure.

All or portions of the methods in accordance to the embodiments may be executed in one or more computing devices including server computers, personal computers, laptop computers, mobile computing devices such as smartphones and tablet computers.

The embodiments may include computer storage media, transient and non-transient memory devices having computer instructions or software codes stored therein, which can be used to program or configure the computing devices, computer processors, or electronic circuitries to perform any of the processes of the present invention. The storage media, transient and non-transient memory devices can include, but are not limited to, floppy disks, optical discs, Blu-ray Disc, DVD, CD-ROMs, and magneto-optical disks, ROMs, RAMs, flash memory devices, or any type of media or devices suitable for storing instructions, codes, and/or data.

Each of the functional units and modules in accordance with various embodiments also may be implemented in distributed computing environments and/or Cloud computing environments, wherein the whole or portions of machine instructions are executed in distributed fashion by one or more processing devices interconnected by a communication network, such as an intranet, Wide Area Network (WAN), Local Area Network (LAN), the Internet, and other forms of data transmission medium.

As used herein, terms “approximately”, “basically”, “substantially”, and “about” are used for describing and explaining a small variation. When being used in combination with an event or circumstance, the term may refer to a case in which the event or circumstance occurs precisely, and a case in which the event or circumstance occurs approximately. As used herein with respect to a given value or range, the term “about” generally means in the range of +10%, +5%, +1%, or +0.5% of the given value or range. The range may be indicated herein as from one endpoint to another endpoint or between two endpoints. Unless otherwise specified, all the ranges disclosed in the present disclosure include endpoints. When reference is made to “substantially” the same numerical value or characteristic, the term may refer to a value within +10%, +5%, +1%, or +0.5% of the average of the values.

The foregoing description of the present invention has been provided for the purposes of illustration and description. It is not intended to be exhaustive or to limit the invention to the precise forms disclosed. Many modifications and variations will be apparent to the practitioner skilled in the art.

The embodiments were chosen and described in order to best explain the principles of the invention and its practical application, thereby enabling others skilled in the art to understand the invention for various embodiments and with various modifications that are suited to the particular use contemplated.

1. A portable automatic microscope system for detecting and classifying plastics and fibers in an environmental and/or industrial water sample, comprising:
a sample introduction module configured to receive an environmental and/or industrial water sample;
a filtration module comprising a membrane holder and at least one membrane filter configured to retain plastics and fibers from the water sample;
a dye application module configured to deliver a dye matrix onto the retained plastics and fibers, wherein the dye matrix comprises at least one lipophilic fluorescent dye and at least one hydrophilic colorimetric dye;
a microscope imaging module, comprising:
an optical system comprising an objective lens and a linear actuator or stepper motor for automatic focus adjustment;
a controllable illumination source configured to provide visible light and ultraviolet light; and
a digital camera configured to capture images of the retained plastics and fibers;
a control module configured to control operation of the sample introduction module, filtration module, dye application module, and microscope imaging module; and
a computing module comprising a processor and memory, the computing module being configured to:
receive the captured images from the digital camera;
identify regions of interest corresponding to plastics and fibers;
extract color, size, and morphology features from the regions of interest; and
classify the plastics and fibers based on the extracted features using an image analysis method;
wherein the system is configured to perform on-site and real-time detection and classification of plastics and fibers in the environmental and/or industrial water sample.

2. The portable automatic microscope system of claim 1, wherein the membrane holder is configured to receive the at least one membrane filter, and at least one membrane filter has a pore size within about 100 μm to about 5000 μm.

3. The portable automatic microscope system of claim 2, wherein the at least one membrane filter is made of PVDF, cellulose acetate, or nylon.

4. The portable automatic microscope system of claim 1, wherein the portable automatic microscope system further comprises a backwash module configured to clean the membrane filter.

5. The portable automatic microscope system of claim 1, wherein the system is operably connected to a computing device having a display, the computing device being configured to allow a user to operate and monitor the system.

6. The portable automatic microscope system of claim 1, wherein the sample introduction module comprises a pump configured to deliver the water sample.

7. The portable automatic microscope system of claim 1, wherein the computing module is further configured to generate and display a quantitative analysis report of detected plastics and fibers.

8. The portable automatic microscope system of claim 1, wherein the computing module comprises a convolutional neural network (CNN) configured to classify plastics and fibers.

9. The portable automatic microscope system of claim 1, wherein the dye matrix is dissolved in a solvent system comprising water and an organic solvent comprising one or more of methanol, ethanol, acetone, and dimethyl sulfoxide (DMSO).

10. A method for detecting and classifying plastics and fibers in an environmental and/or industrial water sample using a portable automatic microscope system, comprising:
introducing an environmental and/or industrial water sample into the system;
filtering the water sample through at least one membrane filter to retain plastics and fibers on the membrane filter;
applying a dye matrix onto the retained plastics and fibers, wherein the dye matrix comprises at least one lipophilic fluorescent dye and at least one hydrophilic colorimetric dye;
illuminating the retained plastics and fibers with visible light and/or ultraviolet light;
capturing one or more images of the retained plastics and fibers using a digital camera;
transmitting the captured images to a computing module;
analyzing the captured images using the computing module to:
identify regions of interest corresponding to plastics and fibers;
extract color, size, and morphology features from the regions of interest; and
classify the plastics and fibers based on the extracted features and generate classification results; and
outputting the classification results identifying types of plastics and/or fibers present in the environmental and/or industrial water sample.

11. The method of claim 10, further comprising a membrane holder configured to receive the at least one membrane filter, and the at least one membrane filter has a pore size within about 100 μm to about 5000 μm.

12. The method of claim 10, wherein the dye matrix is dissolved in a solvent system comprising water and an organic solvent comprising one or more of methanol, ethanol, acetone, and DMSO.

13. The method of claim 10, further comprising adjusting the pH value of the dye matrix to approximately 9-14 before applying the dye matrix to the retained plastics and fibers.

14. The method of claim 10, further comprising heating the dye matrix and retained plastics and fibers to a temperature of 25° C. to 80° C. for 5 to 300 minutes before the illuminating.

15. The method of claim 10, wherein the classifying comprises distinguishing cotton fibers, polyester fibers, polyamide 6, polyamide 66, polypropylene, acrylonitrile butadiene styrene, polymethyl methacrylate, and polyvinyl chloride.

16. The method of claim 10, further comprising cleaning the membrane filter by backwashing after image capture.
